SEQUENCE LISTING (1 sequence)

SEQ ID NO: 1 - DNA, 41 bp, Artificial Sequence
  aptamer sgc8c
atctaactgctgcgccgccgggaaaatactgtacggttaga

(19) United States
(12) Patent Application Publication
(10) Pub. No.: US 2011/0158901 A1
(43) Pub. Date: Jun. 30, 2011

(54) CHITOSAN-BASED NANOPARTICLES AND METHODS FOR MAKING AND USING THE SAME

(76) Inventor: Swadeshmukul Santra, Orlando, FL (US)

(21) Appl. No.: 12/980,775

(22) Filed: Dec. 29, 2010

Related U.S. Application Data
(60) Provisional application No. 61/290,583, filed on Dec. 29, 2009.

(51) Int. Cl. A61K 51/00; A61K 49/00; B29B 9/12; C08L 5/08; B82Y 5/00

(52) U.S. Cl. 424/1.11; 424/9.1; 424/9.6; 264/11; 428/402; 977/773

(57) ABSTRACT

Water-dispersible chitosan-based nanoparticles comprising a cross-linked chitosan polymer are provided. The chitosan-based nanoparticles advantageously have a particle size of about 100 nm or less and may include an imaging agent, a target-specific ligand, and/or a biologically active compound bonded to the chitosan polymer.

### CROSS-REFERENCE TO RELATED APPLICATIONS

This application claims the benefit of U.S. Provisional Patent Application Ser. No. 61/290,583, filed on Dec. 29, 2009, the entirety of which is hereby incorporated by reference.

### STATEMENT OF GOVERNMENT RIGHTS

The work leading to this invention was partly supported by grants from the National Science Foundation (NSF CBET Grant No. 63016011 and NSF-NIRT Grant No. EEC-056560) and the National Institute of Health (Grant No. 2P01HL059412-11A1). Accordingly, the government has certain rights in the invention, as specified by law.

### FIELD OF THE INVENTION

The present invention relates to the field of biological imaging, and more particularly, to chitosan-based nanoparticles and to methods for making and using such nanoparticles.

### BACKGROUND OF THE INVENTION

In recent years, there has been growing interest in developing nanoparticle-based probes for various bioimaging applications, such as for the diagnostic imaging of cancers, the labeling of stem cells, and the imaging of pathogenic cells. Fluorescent quantum dots (Qdots) and dye-loaded silica based nanoparticles, for example, are extensively used in labeling cells and tissue specimens in vitro. These nanoparticle probes are photostable and highly sensitive and even allow real-time imaging of intracellular components. However, applications of these probes have been primarily restricted as such nanoparticles are not biodegradable. Qdots, for example, are cytotoxic due to the presence of heavy metals and silica nanoparticles are not biodegradable, thereby limiting their potential use in biomedical imaging applications.

### SUMMARY OF THE INVENTION

The present inventors have advantageously developed ultra-small chitosan-based (chitosan) nanoparticles, which are highly water-dispersible, and which may be utilized as a substrate for the attachment of imaging agents, target-specific ligands, and/or biologically active molecules. For example, the nanoparticles may have fluorescent labels attached to the chitosan polymer, which may be utilized for various bioimaging applications such as the diagnostic imaging of cancers, the labeling of stem cells, and the imaging of pathogenic cells. Advantageously, the water droplets in the microemulsions described herein serve as nanosized containers, which compartmentalize the chitosan polymer chains (any molecules attached thereto). The particle size remains within a tight range (e.g., 100 nm or less) even with the attachment of various molecules as the particle size is primarily determined by the microemulsion parameters described herein, such as water to surfactant ratio, for example.

In accordance with one aspect of the present invention, there is provided a method for making water-dispersible chitosan-based nanoparticles. The method comprises obtaining a first water-in-oil (W/O) microemulsion comprising an oil, a surfactant, and an aqueous phase having a chitosan polymer. In addition, the method comprises obtaining a second microemulsion comprising an oil, a surfactant, an aqueous phase comprising a carboxyl-group containing compound (e.g., a cross-linker). In one embodiment, the second microemulsion further comprises a coupling agent (e.g. water-soluble EDC that couples amine and carboxyl groups together, forming an amide bond). Further, the method includes reacting components of the first and second microemulsions for a time sufficient to form the chitosan-based nanoparticles and recovering the water-dispersible chitosan-based nanoparticles from the reacted first and second microemulsion components. The water-dispersible chitosan based nanoparticles have an average particle size of 100 nm or less.

In accordance with another aspect of the present invention, there is provided a water-dispersible chitosan-based nanoparticle comprising a cross-linked chitosan polymer having an imaging agent bonded thereto. The chitosan-based nanoparticle has a particle size of 100 nm or less.

In accordance with yet another aspect of the present invention, there is provided an in vivo imaging method. The method comprises administering to a subject a plurality of chitosan-based nanoparticles, wherein at least some of the chitosan-based nanoparticles comprise a chitosan polymer having an imaging agent bonded thereto, and wherein the chitosan-based nanoparticles have an average particle size of 100 nm or less. In addition, the method further comprises detecting a presence of the chitosan nanoparticles.

### BRIEF DESCRIPTION OF THE DRAWINGS

FIG. 1 depicts a TEM image of synthesized fluorescent chitosan nanoparticles (FCNPs) showing nearly uniform particles with average size of 17-28 nm.

FIG. 2 depicts a flow cytometric assay to monitor the binding of sgc8c-conjugated FCNPs to CCRF-CEM cells (target cells) and Ramos cells (control cells). The green (leftmost) curve represents the background binding event of unselected DNA library (lib) conjugated FCNPs and the red (rightmost) curve represents the specific binding event of sgc8c aptamer conjugated FCNPs. For CEM cells, an increase in the binding of sgc8c-conjugated FCNPs is clearly observed, whereas there was no change for the control Ramos cells. These results demonstrated that FCNPs could be specifically targeted to cancer cells.

FIG. 3 represents laser scanning confocal images (left: fluorescence image, right: transmission image) of CCRF-CEM cells labeled by the lib-FCNP (a) and sgc8c-FCNP conjugates (b).

FIG. 4 shows the fluorescent chitosan nanoparticle (FCNP) particle size distribution as determined by Dynamic Light Scattering (DLS) measurements.

FIG. 5 shows the fluorescence excitation (recorded at 519 nm emission) and emission spectra (recorded at 490 nm excitation) of the FCNPs recorded in DI water with characteristic peaks of FITC.

FIG. 6 is a TEM image showing nearly monodispersed bimodal fluorescent and paramagnetic chitosan nanoparticles (BCNPs) with an average size of ˜28 nm. The inset depicts the histogram of particle size distribution.

FIG. 7A-C show fluorescence microscopic images (transmission, 7A and fluorescence, 7B) of J774 cells labeled with BCNPs. FIG. 7C shows T1 weighted images of labeled J774 cells in agar matrix; (i) cell media, (ii) 4×106 unlabeled J774 cells, (in) 1×106 BCNPs labeled J774 cells and (iv) 4×106 BCNPs labeled J774 cells with corresponding T1 values of 3.06 s, 2.44 s, 1.50 s and 1.12 s, respectively.

FIG. 8 shows the fluorescence excitation (recorded at 519 nm emission) and emission spectra (recorded at 490 nm excitation) of FCNPs recorded in DI water with characteristic peaks of FITC.

FIG. 9 shows the bimodal chitosan nanoparticle (BCNP) particle size distribution as determined by Dynamic Light Scattering (DLS) measurements.

FIG. 10 shows a linear plot of Gd concentration versus 1/T₁ to obtain relaxivity r₂ of BCNPs.

FIG. 11 shows a linear plot of Gd concentration versus 1/T₂ to obtain relaxivity r₂ of BCNPs.

### DETAILED DESCRIPTION OF THE INVENTION

In accordance with one aspect of the present invention, there is provided a method for making water-dispersible chitosan-based nanoparticles. The method comprises obtaining a first water-in-oil (W/O) microemulsion comprising an oil, a surfactant, and an aqueous phase having a chitosan polymer. In addition, the method comprises obtaining a second microemulsion comprising an oil, a surfactant, and an aqueous phase having a carboxyl-group containing compound.

In one embodiment, the second microemulsion further comprises a coupling agent, such as EDC. The role of the coupling agent is to couple the carboxyl compounds with amine-group containing chitosan, forming covalently cross-linked chitosan nanoparticles. In another embodiment, where the coupling agent is not used, amine-group containing chitosan (positively charged) can still interact with a carboxyl-containing compound such as dicarboxylic acid and carboxyl-containing polymer (e.g. poly glutamic acid, another type of biodegradable polymer) via electrostatic interaction (Coulombic interaction) without the coupling agent. This type of interaction can be referred as non-covalent cross-linking (or ionic interaction). It is expected that non-covalently cross-linked chitosan particles will degrade faster than covalently cross-linked chitosan nanoparticles.

Further, the method includes reacting components of the first and second microemulsions for a time sufficient to form the chitosan-based nanoparticles and recovering the water-dispersible chitosan-based nanoparticles from the reacted first and second microemulsion components. The water-dispersible chitosan-based nanoparticles have an average particle size of about 100 nm or less. The term “about” as used herein may refer to a value that is ±10% of the stated value.

By “water-in-oil emulsion” as used herein, it is meant that the dispersed phase, water phase in this instance, is a phase consisting of discrete parts fully surrounded by material of another phase, e.g., an oil phase. In addition, as used herein, the terms “chitosan” or “chitosan polymer” refer to chitosan (also known as poliglusam, deacetylchitin, poly-(D)glucosamine) and any derivatives thereof. The chitosan polymer is typically composed of a linear polysaccharide of randomly distributed β-(1-4)-linked D-glucosamine (deacetylated unit) and/or N-acetyl-D-glucosamine (acetylated unit) units. The general terms “chitosan” or “chitosan polymer” as used herein may also refer to chitosan or chitosan having one or more molecules attached thereto, e.g., bonded, or conjugated, thereto, such as an imaging agent, a target-specific ligand, or a biologically active compound.

Exemplary derivatives of chitosan include trimethylchitosan (where the amino group has been trimethylated) or quaternized chitosan. Advantageously, chitosan has a plurality of amine functional groups, which as set forth below, may be utilized for the attachment of various agents thereto, such as imaging agents, target-specific ligands, and biologically active agents.

Chitosan is typically produced by deacetylation of chitin, which is the structural element in the exoskeleton of crustaceans (crabs, shrimp, etc.) and cell walls of fungi. The degree of deacetylation (% DD) can be determined by NMR spectroscopy, and the % DD in chitosan for use in the methods described herein may be in the range of from 20-100%, and typically from 60-100%. In one embodiment, the chitosan as used herein has a molecular weight of from 50,000 to 190,000 daltons. One known method for the synthesis of chitosan is the deacetylation of chitin using sodium hydroxide in excess as a reagent and water as a solvent. This reaction pathway, when allowed to go to completion (complete deacetylation), yields up to 98% product. The amino group in chitosan has a pKa value of ˜6.5, which leads to a protonation in acidic to neutral solution with a charge density dependent on pH and the % DD value. Chitosan is water-soluble, is useful as a bioadhesive, may enhance the transport of polar drugs across epithelial surfaces, is biocompatible, and is critically biodegradable.

As used herein, the term “surfactant” refers to wetting agents that lower the surface tension of a liquid, allowing easier spreading, and lower the interfacial tension between two liquids. Surfactants are typically classified into four primary groups; anionic, cationic, non-ionic, and zwitterionic (dual charge). A nonionic surfactant has no charge groups in its head. The head of an ionic surfactant carries a net charge. If the charge is negative, the surfactant is more specifically called anionic; if the charge is positive, it is called cationic. If a surfactant contains a head with two oppositely charged groups, it is termed zwitterionic. In one embodiment, the surfactant for use in the present invention is a nonionic surfactant. A nonionic surfactant refers to a surfactant in which the hydrophilic head group is uncharged.

In particular embodiments, the surfactant for the first and/or second microemulsions comprises Triton X-100. As used herein, the term “Triton X-100” refers to an octylphenol ethylene oxide condensate (P-octyl polyethylene glycol phenyl ether), available from Union Carbide. The “X” series of Triton detergents are produced from octylphenol polymerized with ethylene oxide. The number (“-100”) relates only indirectly to the number of ethylene oxide units in the structure. X-100 has an average of 9.5 ethylene oxide units per molecule, for example. Alternatively, the surfactant may be any other suitable surfactant material, such as a fatty acid ester, a polyglycerol compound, a polyoxyethylene surfactant, e.g., asBrij-30, Brij-35, Brij-92, Tween-20, and/or Tween-80. In one embodiment, the first and/or second microemulsion also comprises a co-surfactant. The co-surfactant is typically a different surfactant from the primary surfactant used. In one embodiment, the co-surfactant comprises n-hexanol. In another embodiment, the co-surfactant comprises sodium bis(2-ethylhexyl) sulfosuccinate (docusate sodium), also known as Aerosol OT (AOT).

As used herein, the term “oil” refers to any compound that is not miscible with water. Non-limiting examples of suitable oils for use in the present invention, e.g. in the first and second microemulsions, include aliphatic and aromatic hydrocarbons, e.g., hexane, heptane, cyclohexane, toluene and benzene. In one embodiment, the oil comprises cyclohexane.

The carboxyl group-containing compound for the second microemulsion may be any compound comprising one or more carboxylic acid groups. The carboxyl-group containing compound may comprise a monocarboxylic acid or a polycarboxylic acid, for example. In one embodiment, the carboxyl-group containing compound is a dicarboxylic acid. Exemplary dicarboxylic acids include succinic acid, malic acid, aspartic acid, oxalic acid, malonic acid, methyl malonic acid, methyl succinic acid, fumaric acid, 2,3-dihydroxyfumaric acid, tartaric acid, glutaric acid, glutamic acid, adipic acid, pimelic acid, suberic acid, azelaic acid, and sebacic acid.

In a particular embodiment, the carboxylic acid comprises tartaric acid. While not wishing to be bound by theory, it is believed that tartaric acid helps maintain the integrity of the compartmentalized chitosan nanoparticles via tartaric acid-mediated covalent cross-linking. The present inventors have found that covalent cross-linking via tartaric acid is more attractive for making stable chitosan nanoparticles than ionic gelation, for example, which would be compromised in an acidic environment. Further, aside from serving as a biocompatible cross-linker, it is believed that tartaric acid improves hydrophilicity of the chitosan-based nanoparticles by incorporating numerous hydroxyl groups in the polymeric nanoparticle matrix.

In another embodiment, the carboxyl group-containing compound is a polycarboxylic acid, such as a tricarboxylic acid. Exemplary tricarboxylic acids include citric acid, isocitric acid, aconitic acid, and propane-1,2,3-tricarboxylic acid. In yet another embodiment, the carboxyl group-containing compound comprises any other suitable dicarboxylic or polycarboxylic compound. For example, in one embodiment, the carboxyl group-containing compound may comprise a polymer comprising one or more carboxyl groups, such as polyglutamic acid.

The first microemulsion may be prepared by combining the components under stirring conditions for at least a few minutes, e.g., five minutes. In one exemplary embodiment, the first microemulsion is formed by the dropwise addition of Triton X-100 to a mixture of the cyclohexane, n-hexanol, and the chitosan polymer. Upon magnetic stirring for about an hour, a yellow-colored, stable, completely transparent microemulsion may be formed. In a particular embodiment, the first microemulsion may be formed by dropwise addition of Triton X-100 to a mixture of cyclohexane (11 ml), n-hexanol (4 mL) and aqueous phase (4 mL) containing a mixture of FITC-chitosan (2 ml as dialysed) and unlabeled chitosan polymer (2 mL, dialysed). For making dialysed chitosan aqueous solution, 0.25% chitosan polymer solution prepared in 1% acetic acid is allowed to dialyse against DI water for 48 hours. The fluorescent dye labeling efficiency was found to be 3.1 w/w % of FITC to FITC-chitosan polymer.

In addition, in one embodiment, activated tartaric acid for the second microemulsion may be prepared by reacting tartaric acid with N-hydroxysuccinimide (NHS) and 1-ethyl-3-(3-dimethylaminopropyl carbodiimide hydrochloride) (EDC) to activate the tartaric acid. In one exemplary embodiment, the tartaric acid, EDC, and The NHS is a promoter of EDC-based coupling reactions. Without the NHS, the coupling reaction will take place. However, EDC-tartaric acid conjugate may not be fully stable in aqueous solution. In the presence of NHS, EDC-tartaric acid conjugates to NHS ester of tartaric acid, which is quite stable in water and it is amine-reactive. NHS may be combined with tartaric acid and EDC in a ratio of about 1:5:2 (tartaric acid:EDC:NHS) and stirred, e.g., magnetically stirred, for a suitable period of time, e.g., 15 minutes. The second microemulsion may then be prepared by adding a quantity of the activated tartaric acid solution to a Triton X-100/cyclohexane/n-hexanol system and stirring for a few minutes, e.g., five minutes.

Thus, the aqueous phase of the second microemulsion may comprise a mixture of tartaric acid (a cross-linker), a water soluble carbodiimide (a coupling agent, e.g., water-soluble EDC), and N-hydroxy succinimide (NHS). The carbodiimide coupling agent couples (or combines) first the carboxyl group of the tartaric acid with the NHS, forming a stable NHS ester derivative of the tartaric acid. The resulting NHS ester of tartaric acid is amine group reactive. Once combined with the chitosan polymer, the amine group of the chitosan polymer reacts with the NHS ester of the tartaric acid, forming a stable amide (—NHCO—) bond (also called the peptide bond as it is found in peptides, proteins). Tartaric acid molecule has two carboxyl groups and serves as a cross-linker. Therefore each tartaric acid molecule can combine two amine groups of the chitosan polymer, forming two peptide bonds. Thus, the crosslinking process combines both labeled chitosan polymer and unlabeled chitosan polymer together within the microemulsion water droplet, forming covalently crosslinked nanoparticles.

After the first and the second microemulsions are formed, the second microemulsion may be added drop-wise to the first microemulsion and stirred to react components of the first and second microemulsions together to form the chitosan-based nanoparticles. After the addition is finished, the microemulsions may be continuously mixed by stirring for a suitable period of time to ensure a complete reaction. Note that carbodiimide based cross-linking reaction takes place instantly (e.g., water-soluble EDC and NHS react first with tartaric acid within a few minutes and form an amine reactive N-hydroxy succinimide (NHS) ester of tartaric acid. Once the microemulsion containing the chitosan polymer is allowed to react with the microemulsion containing NHS ester of tartaric acid, cross-linking reaction takes place instantly (within a few minutes). A period of about two hours time should be more than sufficient to ensure complete reaction. Dark conditions may be required for experiments that involve fluorescein isothiocyanate (FITC) or iohexyl, otherwise normal room light conditions are typically maintained during stirring.

The formed water-dispersible chitosan-based nanoparticles may be recovered from the reacted first and second microemulsions by any suitable method known in the art. In one embodiment, the formed chitosan-based nanoparticles are recovered after the reacting by the addition of ethanol so as to separate the nanoparticles from the microemulsion. The addition of the ethanol destabilizes the microemulsion system resulting in the precipitation of the nanoparticles from the microemulsion. In one embodiment, the ethanol may comprise a 95% (V/V) ethanol solution. After reacting and recovering the formed chitosan-based nanoparticles, the method may further comprise washing the recovered nanoparticles in ethanol at least once, followed by suspending the recovered nanoparticles in a fluid carrier, such as water. In order to further clean the particle suspension, the suspended recovered nanoparticles may be further dialysed against water. Dialysis is the process of separating molecules in solution by the difference in their rates of diffusion through a semipermeable membrane, such as dialysis tubing.

In one embodiment, in the washing step, the nanoparticles may be pelleted by centrifugation at 8000 rpm in an Eppendorf, model 5810R, angle-head centrifuge, for example, in a 35 ml total volume for 15 minutes. Those skilled in the art will be able to determine centrifugation conditions necessary for pelleting these nanoparticles in other centrifuge systems. Further, in washing, ethanol may be added to the centrifuged nanoparticles followed by vortexing for a few minutes and then sonication (using a sonic bath) for about 10 seconds. This allows nanoparticles to re-disperse uniformly in the ethanol. This ethanol solution may then be centrifuged for 15 minutes. Nanoparticles at this stage typically settle down at the bottom of the centrifuge tube. The supernatant may then be discarded. This washing procedure (addition of ethanol to the centrifuged nanoparticles, vortexing the solution followed by sonication, centrifugation and removal of the supernatant) may be repeated multiple times, e.g., five times. Washed nanoparticles may be resuspended in a fluid carrier, preferably water, and aggregated nanoparticles may be separated from monodispersed nanoparticles by filtration.

In certain aspects of the present invention, the chitosan polymer is labeled with an additional moiety or compound, such as an imaging agent, a ligand having an affinity for a specific target, and a biologically active material to form chitosan-based nanoparticles having such additional moieties or compounds incorporated therein. In one embodiment, the additional compound or moiety is bonded to the chitosan polymer prior to the reacting of the components of the first microemulsion and the second microemulsion, although it is understood that the present invention is not so limited. It is contemplated that the additional compounds or moieties described herein may be bonded to the chitosan polymer by covalent bonding through the amine groups of the chitosan polymer, although the invention is not so limited.

In accordance with one aspect of the present invention, the chitosan polymer is labeled with (attached to) an imaging agent. For example, the imaging agent may comprise one or more of a fluorophore, iohexyl, and a paramagnetic chelate having a paramagnetic ion bound therein. In one embodiment, the chitosan polymer is labeled with a fluorophore. In another embodiment, the chitosan polymer may be labeled with a fluorophore and also a paramagnetic chelate (chelator) having an MRI (magnetic resonance imaging) contrast agent bound therein linked to the chitosan polymer so that the recovered nanoparticles are effective as a bimodal agent that is fluorescent as well as paramagnetic. The MRI contrast agent may comprise a paramagnetic ion selected from one or more of gadolinium, dysprosium, europium, and compounds, or combinations thereof, for example. In another embodiment, the chitosan polymer may be solely or additionally linked with iohexyl such that the recovered nanoparticles are radio-opaque. In one embodiment, the paramagnetic ion is a gadolinium ion and the chelator is a DOTA-NHS ester (2,2′,2″-(10-(2-(2.5-dioxopyrrolidin-1yloxy)-2-oxoethyl)-14,7,10-tetraazacyclododecane-1,4,7-tryl)triaceticacid). Gd³⁺ ions are paramagnetic and DOTA is a chelator of Gd ion. The Gd-DOTA is paramagnetic agent and it gives MRI contrast. Gd-DOTA is commercially available under the brand name ProHance® (also called Gadoteridol)

When a fluorophore is provided, the fluorophore may comprise at least one of fluorescent dye, a quantum dot (Qdot), a bioluminescence agent, or combinations thereof. Exemplary bioluminescent agents include a luciferase enzyme and are described in So, M.-K., Xu, C., Loaning, A. M., Gambhir, S. S. & Rao, J. Nat. Biotechnol. 24, 339-343 (2006), the entirety of which is incorporated by reference herein.

In another embodiment, the chitosan polymer may be labeled with a radioisotope, e.g., a positron emitting radio-isotope (such as ³¹P, ¹¹C, ¹⁸F etc) for PET imaging or a gamma emitting radio isotope (such as ^{99m}Tc, ¹¹¹In, ¹²³I and ¹⁵³Sm) for detection using gamma camera. See Perkins, A. C. and M. Frier, Radionuclide imaging in drug development. Current Pharmaceutical Design, 2004. 10(24): p. 2907-2921; Longjiang Zhang, Hongwei Chen, Liya Wang, Tian Liu, Julie Yeh, Guangming Lu, Lily Yang, Hui Mao; Delivery of therapeutic radioisotopes using nanoparticle platforms: potential benefit in systemic radiation therapy. Nanotechnology, Science and Applications, 2010, Volume 2010:3, p 159-170), the entireties of which are incorporated by reference herein.

In other embodiments, the chitosan polymer may be solely or additionally labeled with a target-specific ligand (target molecule) attached, bonded, or otherwise linked to the chitosan polymer, wherein the ligand has an affinity for a predetermined molecular target. Again, as with any other additional agent that may be attached to the chitosan polymer, the target-specific ligand may be attached to the chitosan polymer before combination with the second microemulsion, e.g., added to the aqueous phase of the first microemulsion prior to combination with the remaining components of the first microemulsion. The target-specific ligand may be one or more of an aptamer, a peptide, an oligonucleotide, folic acid, an antigen, an antibody, or combinations thereof. In one embodiment, the predetermined molecular target is associated with a cancer cell, a leukemia cell, an acute lymphoblastic leukemia T-cell, or combinations thereof.

In a particular embodiment, the target-specific ligand is folic acid, which has a known affinity for cancerous cells, such as breast cancer cells. In another embodiment, the ligand comprises an aptamer having an affinity for leukemia cells, e.g., an acute lymphoblastic leukemia T-cell. As used herein, the term “aptamer” refers to any oligonucleic acid or peptide molecules that bind to a specific target molecule. The aptamer may include any polynucleotide- or peptide-based molecule. A polynucleotidal aptamer is a DNA or RNA molecule, usually comprising several strands of nucleic acids that adopt highly specific three-dimensional conformation designed to have appropriate binding affinities and specificities towards specific target molecules, such as peptides, proteins, drugs, vitamins, among other organic and inorganic molecules. Such polynucleotidal aptamers can be selected from a vast population of random sequences through the use of systematic evolution of ligands by exponential enrichment. A peptide aptamer is typically a loop of about 10 to about 20 amino acids attached to a protein scaffold that bind to specific ligands. Peptide aptamers may be identified and isolated from combinatorial libraries, using methods such as the yeast two-hybrid system. In one embodiment, the ligand comprises the DNA aptamer sgc8c having a sequence according to SEQ. ID No. 1:

5′-ATC TAA CTG CTG CGC CGC CGG GAA AAT ACT GTA
CGG TTA GA-3′.

The DNA aptamer sgc8c has been shown to have a particular binding affinity for leukemia cells, e.g, acute lymphoblastic leukemia T-cells.

In still other embodiments, a biologically active material may be bonded to the chitosan polymer. Exemplary biologically active materials include peptides (e.g., RGD peptide, integrin selective; see Dechantsreiter, M. A., at al., N-Methylated Cyclic RGD Peptides as Highly Active and Selective αᵥβ₃ Integrin Antagonists, Journal of Medicinal Chemistry, 1999. 42(16): p. 3033-3040), antibodies (e.g., CD10 monoclonal antibody for targeting human leukemia; see Santra, S., at al., Conjugation of Biomolecules with Luminophore-Doped Silica Nanoparticles for Photostable Biomarkers. Analytical Chemistry, 2001. 73(20): p. 4988-4993.) and proteins. The nanoparticles of the present invention may be employed as biologic agents in that, for example, the chitosan polymer may be conjugated with a ligand having an affinity for a predetermined biological target so that nanoparticles are effective as target-specific probes. Likewise, the chitosan polymer may be conjugated with a biologically active drug, as well as with a target-specific ligand. When these two modalities are combined, the disclosed nanoparticles are useful as target-specific drug delivery vehicles.

The above-described methods are capable of producing water-dispersible chitosan nanoparticles comprising at least cross-linked chitosan polymer and typically an imaging agent bonded thereto. Advantageously, the formed water dispersible chitosan-based nanoparticles advantageously have an average particle size of 100 nm or less, and in one embodiment, about 60 nm or less, and in another embodiment, from about 15-35 nm. In one embodiment, the stated values refer to a longest dimension of the particle. It is appreciated that larger nanoparticles may be formed, e.g., 200 nm, upon agglomeration of two or more nanoparticles. Nanoparticles having a particle size of about 100 nm or less have several advantages; (i) due to large surface to volume ratio, it is possible to co-attach targeting molecules, image contrast agents and/or therapeutic drugs to the nanoparticle surface as described herein; (ii) the chitosan nanoparticles may be capable of evading the macrophage capture of the immune system and may remain in the circulation for a longer time for effective therapy, (iii) intra-cellular delivery of the chitosan nanoparticles may be facilitated; and (iv) the chitosan nanoparticles may easily travel through the smallest blood capillary (5-6 microns in diameter) without forming embolism, allowing uniform distribution in the circulation.

Furthermore, the present inventors have found that particle size does not typically change irrespective of whether there a single-modal chitosan-based nanoparticle (such as an FITC-labeled chitosan-based pnanoparticle) or a bi-modal (both FITC and Gd-DOTA labeled) chitosan-based nanoparticle. This indicates that particle size depends on microemulsion parameters such as a water to surfactant molar ratio, which may be from 2 to 70 and in a particular embodiment about 10:1. See Padmavathy Tallury, Soumitra Kar, Suwussa Bamrungsap, Yu-Fen Huang, Weihong Tan and Swadeshmukul Santra, Chem. Commun., 2009, 2347-2349. It is appreciated that the ratio may be as high as 70:1 in case of the AOT-based water-in-oil microemulsion systems. See Ref. De, T. K. and A. Maitra, Solution behaviour of Aerosol OT in non-polar solvents. Advances in Colloid and Interface Science, 1995. 59: p. 95-193 and reference #94 cited therein.

In addition, in one embodiment, the chitosan-based nanoparticles have a zeta potential of at least +28 mV. Zeta (ζ) potential is a parameter characterizing electric properties of interfacial layers in dispersions, emulsion, porous bodies. The positive zeta potential of the formed nanoparticles likely indicates the presence of surface amine functional groups. The zeta potential provides information about a nanoparticle's surface charge. For example, positively charged nanoparticles may have good transfecting capability whereas negatively charged particles should have minimal or no transfecting capability. For drug delivery applications (non-targeted), it is desirable to have positively charged particle based drug carriers.

In accordance with another aspect of the present invention, there is provided an in vivo imaging method. The method comprises administering to a subject a plurality of chitosan-based nanoparticles, wherein at least some of the chitosan nanoparticles comprise a chitosan polymer having an imaging agent bonded thereto, and wherein the chitosan-based nanoparticles have an average particle size of about 100 nm or less. In addition, the method further comprises detecting a presence of the chitosan nanoparticles.

The administering may be done according to any suitable route of in vivo administration that is suitable for delivering the composition into a patient (e.g., human or animal subject). The preferred routes of administration will be apparent to those of skill in the art, depending on the medium and/or the predetermined molecular target. Exemplary methods of in vivo administration include, but are not limited to, intravenous administration, intraperitoneal administration, intramuscular administration, intranodal administration, intracoronary administration, intraarterial administration (e.g., into a carotid artery), subcutaneous administration, transdermal delivery, intratracheal administration, intraarticular administration, intraventricular administration, inhalation (e.g., aerosol), intracranial, intraspinal, intraocular, intranasal, oral, bronchial, rectal, topical, vaginal, urethral, pulmonary administration, impregnation of a catheter, and direct injection into a tissue. The detecting may be done by any suitable detection method known in the art appropriate for the particular type of imaging agent incorporated into the chitosan-based nanoparticles. For example, the detection may be done by fluorescence spectroscopy in the case of a fluorophore.

In one embodiment, the imaging agent for the method comprises a fluorophore, a a paramagnetic chelate having a paramagnetic ion bound therein, or both. In addition, the chitosan-based nanoparticles may further include a target-specific ligand bonded to the chitosan polymer, wherein the ligand is specific for a predetermined molecular target.

The following examples are intended for the purpose of illustration of the present invention. However, the scope of the present invention should be defined as the claims appended hereto, and the following examples should not be construed as in any way limiting the scope of the present invention.

### Example 1

In this example, fluorescent chitosan nanoparticles (FCNPs) were synthesized using a homogeneous water-in-oil (W/O) microemulsion system consisting of cyclohexane (oil), Triton X-100 (surfactant), n-hexanol (co-surfactant) and water. To retain the particulate integrity, the FCNPs were covalently cross-linked with tartaric acid. Water-insoluble low molecular weight (50-190 kDa) chitosan polymer was first dissolved in 1% acetic acid solution. A part of the chitosan solution was treated with excess amount of amine-reactive fluorescent dye, fluorescein isothiocyanate (FITC), which produced FITC labeled chitosan polymer. Unbound FITC molecules were removed by ethanol/water washing. For the FCNP synthesis, two separate water-in-oil microemulsions, ME and ME U, were prepared. The aqueous phase of ME I contained a mixture of FITC labeled chitosan and pure chitosan polymer solutions. The ME II aqueous phase comprised of a mixture of the crosslinker tartaric acid and water-soluble carbodiimide. Both aqueous phases were combined with the cyclohexane/Triton X-100/n-hexanol system. The FCNPs were then produced by simply adding ME II to ME I, followed by overnight magnetic stirring. Once ME II is added to ME I, water droplets (aqueous phase) from both MEs are mixed (a process called coalescence) and hence aqueous phase components (see Santra, S., et al., Conjugation of Biomolecules with Luminophore-Doped Silica Nanoparticles for Photostable Biomarkers. Analytical Chemistry, 2001. 73(20): p. 4988-4993). By adding ethanol, the yellow colored FCNPs were precipitated from the microemulsion and thoroughly washed.

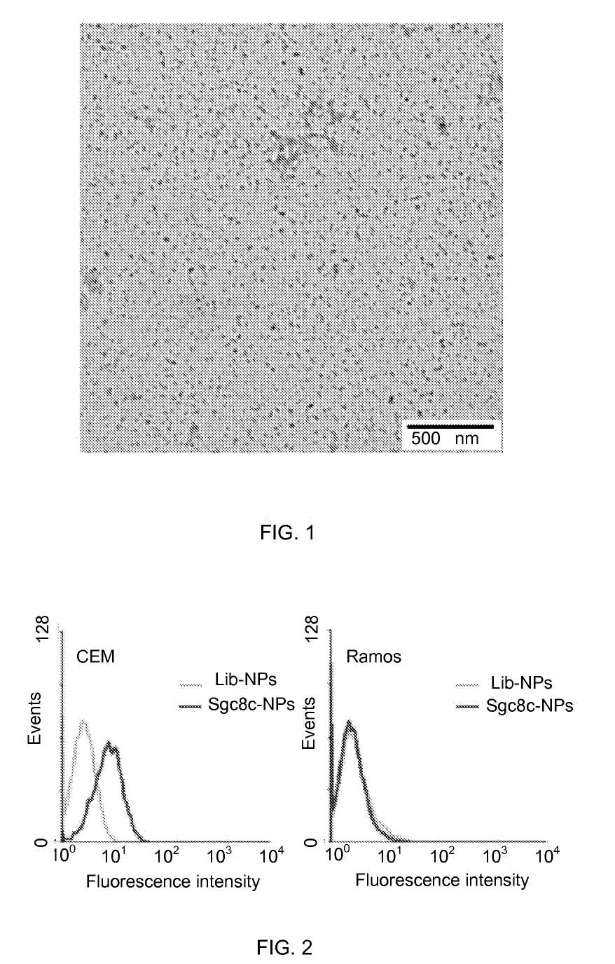

The FCNPs were uniform in size as measured by the transmission electron microscopy (TEM) and the average particle size was 28 nm based on the average of 50 particles (FIG. 1). The formation of ultra-small size FCNPs may be due to the compartmentalization of chitosan polymer within the 10-15 nm size microemulsion water droplet that served as a “nano-container.” (Note that droplet size can increase when water droplets contain polymer molecules). The compartmentalization process was likely induced by several factors such as the confined environment of the nano-container and partial neutralization of the protonated primary amine groups (“charge shielding”) of the chitosan polymer due to interaction with the surfactant and co-surfactant molecules at the oil/water interface. The integrity of the compartmentalized FCNPs was maintained via tartaric acid-mediated covalent cross-linking. Tartaric acid played a dual role. Besides serving as a biocompatible cross-linker, the tartaric acid drastically improved hydrophilicity of the FCNPs by incorporating numerous hydroxyl groups in the polymeric nanoparticle matrix. Zeta potential measurement of the FCNPs showed positive surface charge with zeta potential value of +28 mV, confirming the presence of surface amine groups. The superior water solubility of the FCNPs could be explained on the basis of increased hydrophilicity (highly solvated), ultra-small particle size (minimal effect of gravity) and electrostatic force of repulsion (charge stabilization). Dynamic light scattering (DLS) measurements showed particle size distribution in DI water in the ranges of 38 nm and 197 nm. The wide size distribution is primarily due to self-adhesive nature of chitosan polymer as reported in the literature that induces particle-particle association. The fluorescence excitation (recorded at 519 nm emission) and emission spectra (recorded at 490 nm excitation) of the FCNPs recorded in DI water showed characteristic peaks of FITC, indicating that the chitosan matrix did not alter photophysical properties of the FITC molecules.

### Example 2

Identifying cancer is crucial for its early detection and diagnosis. To facilitate this, DNA aptamers specific to cancer biomarkers have become effective specific molecular probes and their conjugation to nanoparticles has given a new dimension to diagnostic and therapeutic applications. To demonstrate specific targeting to the CCRF-CEM cells (CCL-119 T-cell, human acute lymphoblastic leukemia), the FCNPs described above were covalently attached to the DNA aptamer sgc8c (5′-ATC TAA CTG CTG CGC CCC CGG GAA AAT ACT GTA CGG TTA GA-3′). The sgc8c is a strong aptamer with K_{d} in the nM range. The aptamer was modified at the 5′ position with a carboxyl group. The carboxylated aptamer was then conjugated to the surface amine groups of the FCNPs using water soluble carbodimide chemistry. Similarly, as a negative control, a library of randomized sequence of ssDNA 41 nucleotides was conjugated to the FCNP surface. The aptamer conjugated FCNPs were incubated with CCRF-CEM cells and Ramos cells (CRL-1596, B-cell, human Burkitt's lymphoma).

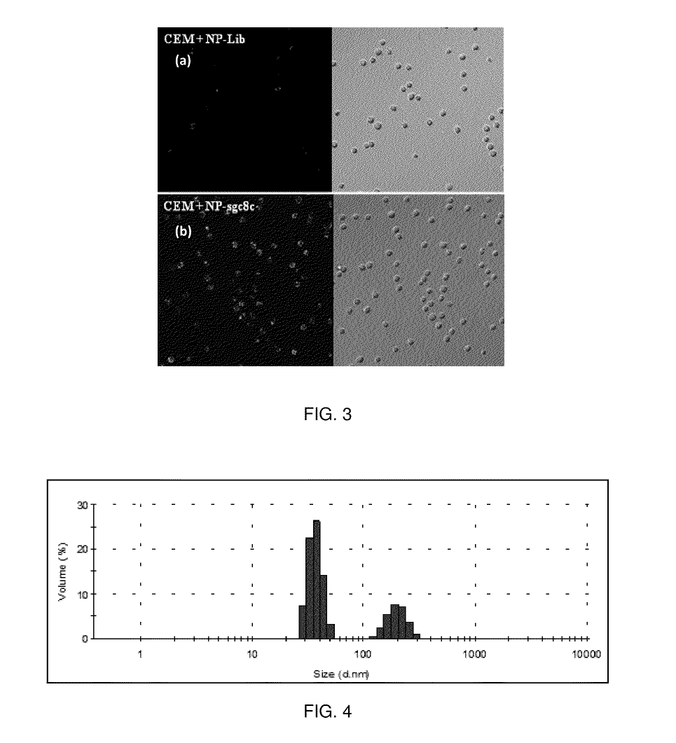

About 1×10⁶ of each cell type was mixed with 100 μL of 0.05 mg/mL concentration of the nanoparticles and incubated on ice for 20 min or 3 hours. After incubation, the cells were washed twice by centrifugation with 0.5 mL of buffer and re-suspended in 0.2-mL volume of buffer. FIG. 2 shows the flow cytometric comparison of target (CCRF-CEM) cells and control negative (Ramos) cells after 20-min incubation with both the negative control and sgc8c conjugated FCNPs. A noticeable change in the fluorescence signal was observed with sgc8c-FCNP labeled CCRF-CEM cells when compared to the negative control, indicating that the binding capability of the aptamer probes was maintained well after the conjugation with FCNPs. No significant change in fluorescence intensity on Ramos cells was observed, further confirming the specific recognition of the aptamer-NP conjugates for target cells. Confocal images as shown in the FIG. 3 further confirmed specific recognition of sgc8c conjugated FCNPs to the CCRF-CEM cells with respect to appropriate controls.

### Example 3

Example 3 more particularly describes a procedure for making FITC-labeled chitosan nanoparticles set forth in Example 1. Low molecular weight chitosan polymer (75-85% deacetylated), Triton X-100, N-hydroxysuccinimide (NHS), 1-ethyl-3-(3-dimethylaminopropyl carbodiimide hydrochloride) (EDC) were purchased from Sigma-Aldrich Chemical Co., USA; Fluorescein isothiocyanate (FITC), anhydrous ethanol were purchased from Fisher Scientific. Dialysis cellulose membrane (MWCO, 6-8 kD) was purchased from Spectrum Laboratories (Rancho Dominguez, Calif.). Deoxyribonucleotides and 5′-carboxyl modifiers were purchased from Glen Research (Sterling, Va.). All solvents and reagents were obtained from Fisher Scientific and were used without further purification. CCRF-CEM cells (CCL-119 T-cell, human acute lymphoblastic leukemia) and Ramos cells (CRL-1596, B-cell, human Burkitt's lymphoma) were obtained from American Type Culture Association, USA. All of the cells were grown in RPMI-1640 containing 10% fetal bovine serum (FBS) and 100 IU/mL penicillin-Streptomycin at 37° C. in a humid atmosphere with 5% CO₂. G25 Sephadex size-exclusion column (NAP™-5) was procured from Amersham Pharmacia Biotech, USA.

The degree of deacetylation of chitosan was determined by elemental analysis at the Atlantic Microblabs, Norcross, Ga. A JEOL JEM 1011 100 kV transmission electron microscope (TEM) was used to characterize particle size. TEM sample was prepared by placing a drop of the chitosan nanoparticles on a carbon coated copper grid (400 mesh size) followed by air drying. Particle size distribution and zeta potential in solution was measured by the Dynamic Light Scattering (DLS) using Malvern Zeta Sizer (model: NanoZS). The concentration of activated aptamer was determined by UV-Vis spectrophotometer (Cary 100, Varian, Inc., CA), Fluorescence excitation and emission spectra were recorded on SPEX Nanolog (HORIBA Jobin Yvon) spectrofluorometer. Flow cytometric analysis was carried out in FACScan cytometer (Becton Dickinson Immunocytometry Systems, San Jose, Calif.). Fluorescence imaging was conducted with a confocal microscope setup consisting of an Olympus IX-81 inverted microscope with an Olympus Fluoview 500 confocal scanning system.

The synthesis of FITC labeled chitosan (FITC-Chitosan) polymer was performed as follows. The FITC is an amine reactive fluorescent dye. The isothiocyanate group readily reacts with the primary amine groups of the chitosan polymer. The covalent attachment of the FITC to the chitosan polymer was carried out as follows. First, 0.25% chitosan polymer solution was prepared in 1% acetic acid solution. Second, 6 mL of the chitosan polymer solution was treated with excess amount of FITC (dissolved in 6 ml of anhydrous ethanol, purged with N₂ gas) where the primary amine to FITC ratio was about 1:1.5. Under magnetic stirring condition, the reaction was allowed to continue for about a couple of hours in dark. Third, about 10 ml of 0.1 M NaOH was added to the reaction mixture to precipitate the FITC labeled chitosan polymer. Fourth, the precipitated FITC-chitosan polymer was centrifuged and washed repeatedly with a mixture of ethanol/water (70:30) till the washings were free of FITC (checked by the fluorescence measurements). Finally, the FITC labeled chitosan polymer was dissolved in 1% acetic acid and dialyzed against deionized water for about 48 hours.

The synthesis of FITC-labeled chitosan nanoparticles (FCNP) was carried out using TritonX-100/cyclohexane/n-hexanol/water in a water-in-oil (W/O) microemulsion system. The cross-linker or cross-linker used was 25% stoichiometric ratio of tartaric acid. The carboxyl group of the dicarboxylic acid was reacted to the amine groups of the chitosan by water soluble carbodiimide chemistry at room temperature. In a typical procedure, two separate W/O microemulsions (ME I and ME II) were prepared. ME I was formed by dropwise addition of Triton X-100 to a mixture of cyclohexane (11 ml), n-hexanol (4 mL), a mixture of FITC-chitosan (2 ml as dialysed) and unlabeled chitosan polymer (2 mL). Upon magnetic stirring for about an hour, a stable yellow-colored completely transparent microemulsion was formed. The ME II consisted of the activated tartaric acid cross-linker. The activation of tartaric acid was done following traditional water-soluble carbodiimide coupling agent, EDC, where tartaric acid, EDC and NHS were combined in a ratio of 1:5:2 and reacted for 15 minutes. ME II was formed by dropwise addition of neat Triton X-100 to a mixture of cyclohexane (11 ml), n-hexanol (4 mL), and the activated tartaric acid solution (4 mL). The ME II was then added dropwise to ME I under magnetic stirring and the crosslinking reaction was continued for 24 hours at room temperature. The FITC labeled covalently cross-linked chitosan nanoparticles were then collected upon breaking the microemulsion system by adding ethanol followed by centrifugation. The yellow colored nanoparticles were washed repeatedly (6 times) with ethanol. Brief sonication and vortexing were applied during particle washing. About 3 ml of DI water was added to the centrifuged nanoparticle. Nanoparticles remained completely dissolved in DI water. To further remove any trace amount of surfactants and other reagents, nanoparticles were dialyzed against DI water for 48 hours. The dialyzed nanoparticle solution was then filtered using a 0.25 μm syringe filter, wrapped with aluminum foil and stored under refrigeration. The nanoparticle solution was freeze-dried and the yield was calculated to be 10.5 mg/mL. The freeze-dried sample is easily soluble in DI water.

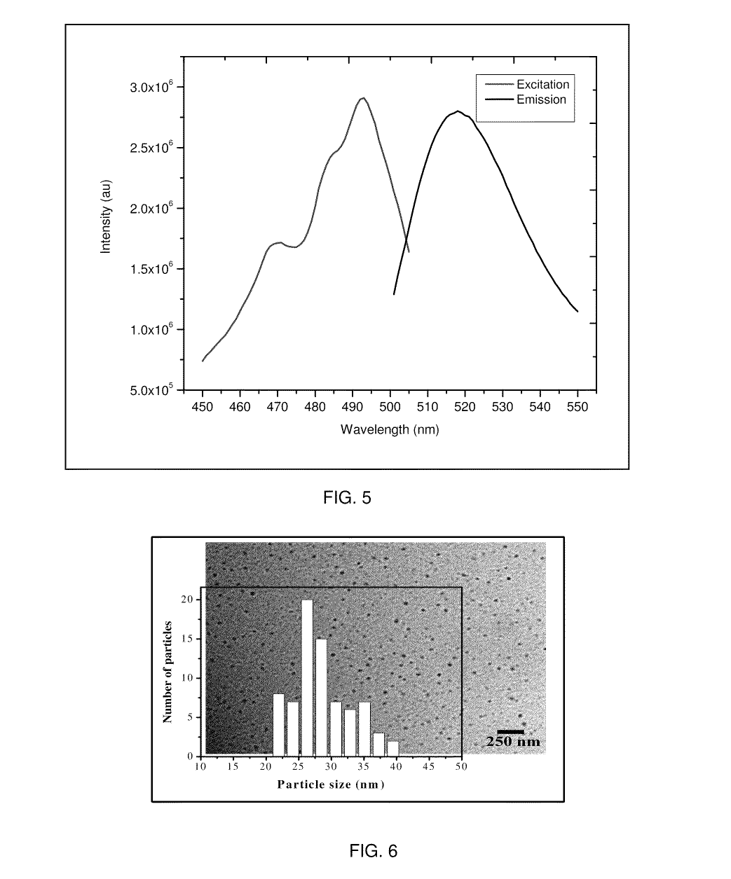

The FCNP particle size distribution was determined by Dynamic Light Scattering (DLS) measurements. FIG. 4 shows the particle size distribution in DI water in the ranges of 38 nm and 197 nm. The excitation and emission spectra of the FITC moiety in the FCNPs was determined by the spectrofluorometer. FIG. 5 shows the fluorescence excitation (recorded at 519 nm emission) and emission spectra (recorded at 490 nm excitation) of the FCNPs recorded in DI water showed characteristic peaks of FITC.

### Example 4

The following example more particularly describes a method for the synthesis of aptamer-chitosan nanoparticle conjugates introduced in Example 2. The aptamers selected were sgc8c, 5′-ATC TAA CTG CTG CGC CGC CGG GAA AAT ACT GTA CGG TTA GA-3 and a library containing a randomized sequence of 41 nucleotides was used as a control. Both the aptamers were coupled with 5-carboxyl modifier. The conjugation was carried out by adding 0.4 mg of EDC (˜2 mM) and 1.1 mg of sulfo-NHS (˜5 mM) to 100 μL of 5 μM carboxyl modified aptamer in 10 mM MES buffer (pH 6.5) and reacted for 30 minutes at room temperature. The excess reagents were separated from the activated aptamer by G25 Sephadex size-exclusion column equilibrated with 10 mM of phosphate buffer (pH 7.4). The concentration of activated aptamer was determined by UV-Vis spectrophotometer, followed by addition of activated aptamer to 0.1 mg/mL of FITC labeled chitosan nanoparticles at a final concentration of 0.05 mg/mL. The mixtures were then incubated for 3 hours at room temperature.

To demonstrate the targeting capabilities of aptamer-conjugated chitosan nanoparticles towards specific cells, fluorescence measurements were made using a FACScan cytometer. About 1×10⁶ of each cell type was mixed with 100 μL of the nanoparticles and incubated on ice for 20 min. After incubation, the cells were washed twice by centrifugation with buffer of 0.5 mL and resuspended in 0.2-mL volume of buffer. The fluorescence was determined by counting 10,000 events. The unselected ssDNA library conjugated with chitosan nanoparticles was used as a negative control.

For confocal imaging, the treatment steps for cell incubation were the same as described for the flow cytometric analysis above. Ten microliters of cell suspension bound with aptamer-conjugated chitosan nanoparticles were dropped on a thin glass slide placed above a 60× objective on the confocal microscope and then covered with a coverslip.

### Example 5

The following example shows a method of fabricating water-soluble small (<30 nm) bimodal (fluorescent and paramagnetic) chitosan nanoparticles (BCNPs) that exhibit bright fluorescence and exceptionally high longitudinal magnetic resonance (MR) relaxivity (41.1 mM Gd⁻¹ s⁻¹). The bimodal (fluorescent and paramagnetic) chitosan nanoparticles (BCNPs) were fabricated as follows.

The first step involved separate labeling of chitosan polymer with amine reactive (a) fluorescein isothiocyanate (FITC), a fluorescent dye and (b) DOTA-NHS ester (1,4,7,10-Tetraazacyclododecane-1,4,7,10-tetraacetic acid mono(N-hydroxysuccinimide ester), a gadolinium ion chelator. The DOTA labeled chitosan was treated with excess gadolinium acetate to obtain Gd-DOTA labeled chitosan polymer. In the second step, two separate water-in-oil microemulsions (ME I and ME II) comprising cyclohexane (oil)/triton X-100 (surfactant)/n-hexanol (co-surfactant)/water were prepared. The aqueous phase of ME I was comprised of both labeled (FITC-chitosan, Gd-DOTA-chitosan) and unlabeled chitosan polymer while the ME II was comprised tartaric of acid cross-linker (activated by the water-soluble reagents, 1-ethyl-3-(3-dimethylaminopropyl carbodiimide hydrochloride. EDC and N-hydroxysuccinimide, NHS). ME II was formed by dropwise addition of neat Triton X-100 to a mixture of cyclohexane, n-hexanol, and the activated tartaric acid solution until the solution becomes transparent. In the final step, the ME II was added dropwise to ME I under magnetic stirring. The BCNPs were recovered from the microemulsion by adding ethanol followed by repeated washing with ethanol to ensure removal of surfactant molecules. The BCNPs remained completely dispersible in DI water.

FIG. 6 is a TEM image showing nearly monodispersed BCNPs with an average size of ˜28 nm. The inset depicts the histogram of particle size distribution. TEM characterization confirmed that BCNPs were nearly monodispersed with average size of 28 nm. The dynamic light scattering (DLS) measurements estimated the particle size to be ˜60 nm which was higher than TEM measurements. This could be attributed to particle-particle association in solution which is reported in the literature. The zeta potential (ζ) measurement of BCNPs showed positive surface charge of +27.6 mV, confirming the presence of surface amine groups. The BCNPs are fluorescently bright showing 520 nm FITC emission upon excitation at 490 nm. Longitudinal (T₁) and transverse (T₂) proton relaxation times were determined as a function of BCNP-Gd concentration at 4.7 T (Tesla). Increased MR signal intensity is observed with increasing Gd concentrations, due to the shorter water T₁ value. The longitudinal relaxivity (r₁) value of 41.12 mM⁻¹ s⁻¹ was determined based on the linear relationship of 1/T₁ plotted against mM Gd concentration. The transverse proton relaxivity (r2) was 111.71 mM⁻¹ s⁻¹ on a per millimolar Gd basis. The high proton relaxivity exhibited by BCNPs is attributed to the following possible factors: (i) enhanced population of water molecules close to paramagnetic center (e.g., “second coordination sphere” relaxation mechanism). Besides the inner sphere (water molecule bound to Gd ions) and the outer sphere (bulk water) contribution towards proton relaxivity, hydrated polymeric nano-environment around Gd-DOTA with increased number of water molecules could have contributed towards “second coordination sphere” relaxation in BCNPs and (ii) slow tumbling rate. In BCNPs, the Gd-DOTA moiety is attached to the rigid and hydrated polymeric environment where water molecules are hydrogen bonded to DOTA carboxyl groups as well as polymeric hydrophilic groups such as amines and hydroxyls. This restriction of free rotation of Gd-DOTA thus could be a contributing factor towards high relaxivity.

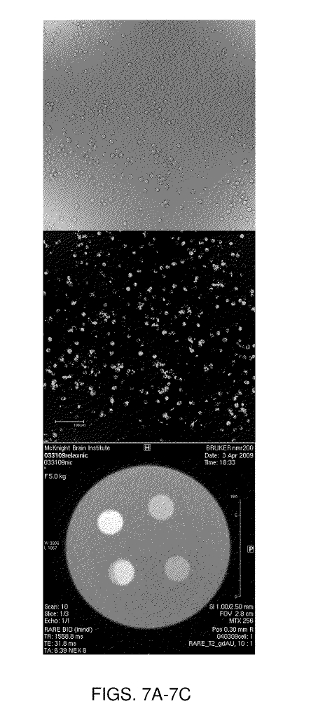

FIG. 7 shows (a) fluorescence microscopic images (transmission, 7A and fluorescence, 7B) of J774 cells labeled with BCNPs. FIG. 7C shows T₁ weighted images of labeled J774 cells in agar matrix; (i) cell media, (ii) 4×10⁶ unlabeled J774 cells, (iii) 1×10⁶ BCNPs labeled J774 cells and (iv) 4×10⁶ BCNPs labeled J774 cells with corresponding T₁ values of 3.06 s, 2.44 s, 1.50 s and 1.12 s, respectively.

Macrophages are shown to aggressively uptake micron/sub-micron size particulates, cellular debris and pathogens. In this example, macrophages were used as an in vitro model system to study non-targeted cellular uptake efficiency of BCNPs. In general, positively charged particles will have strong tendency to remain associated with the cell membrane, consisting of negatively charged phospholipid bilayer (“non-specific binding”). The BCNPs are positively charged but smaller in size (˜60 nm in solution). It was expected that BCNPs will be mostly adhered to cell membrane while some particles will be internalized. Representative fluorescence microscopic (confocal) images of mouse monocyte/macrophage J774 cells labeled with BCNPs are shown in FIG. 7A (transmission) and FIG. 7B (fluorescence). Fluorescence image confirmed high macrophage labeling efficiency. To confirm BCNP internalization, cells were co-labeled with both BCNPs and a carbocyanine membrane dye, DiL. As expected, some BCNPs were found within macrophages (see inset of FIG. 7B. Due to high cell labeling, significant MR contrast was clearly visualized (FIG. 7C) from BCNPs labeled J774 cells with respect to controls (unlabeled J774 cells as well as cell media). Thus, in vitro cell uptake studies against J774 cells clearly demonstrated high macrophage labeling efficiency. As such, these BCNPs could be used to label stem cells for bimodal imaging purposes and could serve as an attractive alternative to cytotoxic quantum dot based bimodal imaging probes.

### Example 6

This example more particularly describes the process for synthesizing the particles introduced in Example 5. Chitosan polymer (low molecular weight), was purchased from Sigma-Aldrich Chemical Co., USA (manufacturer provided chitosan molecular weight 50-190 KDa and degree of deacetylation 75-85%). The viscosity average molecular weight was determined to be 5.3×10⁵ Daltons by Ubbelohde viscometer and the degree of deacetylation was estimated to be 77% by elemental analysis (Tallury et al., Chemical Communications 2009, 17, 2347-2349). A chitosan polymer solution of 0.25% (w/v) prepared in 1% acetic acid solution (w/v) was used for all the following experiments.

Labeling of the chitosan polymer with FITC was carried out as per Chemical Communications 2009, 1, 2347-2349, the entirety of which is incorporated by reference. Synthesis of Gd-DOTA conjugated chitosan polymer (Gd-DOTA chitosan) DOTA (1,4,7,10-Tetraazacyclododecane-1,4,7,10-tetraacetic acid) was covalently attached to the chitosan polymer by the reaction of the NHS functional group of DOTA-NHS ester (Macrocyclics, USA) with the amine groups of chitosan polymer.

Gadolinium acetate hydrate (Aldrich, USA) was added to the above polymer to obtain Gd-DOTA chitosan polymer. Here, the amount of DOTA-NHS ester was varied with respect to the amine groups of the chitosan in the ratios (1:3) (I), (1:5) (II) and (1:7) (III) respectively. Typically, the required amount of DOTA-NHS ester was reacted with 6 ml of 0.25% chitosan solution at room temperature for 24 hours. To this, four times excess of gadolinium (III) acetate in water was added and reacted for 24 hours. To remove excess of Gd ion, excess amount of EDTA was added. The Gd-DOTA-chitosan polymer was purified via dialysis against DI water for 48 hours. The dialyzed Gd-DOTA-chitosan conjugate (I-III) were measured for longitudinal relaxation time T₁ using a 0.5 T Minispec Bruker spectrometer. The T₁ values measured are presented below:

| Solution | DOTA-NHS:Chitosan-NH2 | T1 (ms) |
|---|---|---|
| Gd-DOTA-chitosan conjugate I | 1:3 | 126 |
| Gd-DOTA-chitosan conjugate II | 1:5 | 74 |
| Gd-DOTA-chitosan conjugate III | 1:7 | 76 |
| DI water | — | 2500 |

It was observed that with decreasing concentration of DOTA-NHS with respect to chitosan, T₁ relaxation times shortened. Therefore, we used as dialyzed Gd-DOTA-chitosan conjugate III that used less DOTA for BCNP synthesis. As dialyzed solution of Gd-DOTA polymer III was used for making the BCNPs.

Synthesis of FITC and Gd-DOTA co-labeled ultra-small chitosan nanoparticles (bimodal chitosan nanoparticles (BCNPs)) was carried out using TritonX-100/cyclohexane/n-hexanol/water microemulsion system. A stoichiometric ratio of 25% (to the amine groups of chitosan polymer) tartaric acid (Bodnar et al, Biomacromolecules, 6, 2521, 2004) was used a crosslinker in nanoparticle synthesis. The carboxyl groups of tartaric acid were reacted with the amine groups of the chitosan using carbodiimide coupling chemistry. In atypical procedure, two separate water-in-oil (W/O) microemulsions (ME I and ME II) were prepared.

The ME I was formed by dropwise addition of Triton X-100 to a mixture of cyclohexane (11 ml), n-hexanol (4 mL), a mixture of FITC-chitosan, Gd-DOTA chitosan and unlabeled chitosan polymer (4 mL). A stable yellow-colored transparent microemulsion was obtained after stirring for 1 h. The ME II consisted of the activated tartaric acid crosslinker as the aqueous phase. The activation of tartaric acid was done separately by reacting tartaric acid, EDC and NHS in 1:5:2 ratio for 15 minutes (Damink L et al, Biomaterials, 17, 765, 1996). ME II was formed by dropwise addition of neat Triton X-100 to a mixture of cyclohexane (11 ml), n-hexanol (4 mL), and the activated tartaric acid solution (4 mL) until the solution becomes transparent. The ME II was then added dropwise to ME I and stirred for 24 h. The covalently crosslinked BCNPs were then collected by breaking the microemulsion system with ethanol followed by centrifugation. The yellow colored nanoparticles were washed repeatedly (8 times) with ethanol. The nanoparticles were subjected to brief sonication and vortexing during washing steps. About 3 ml of DI water was added to the centrifuged nanoparticles in which they were easily dispersed resulting in a transparent homogeneous solution. The concentration of the nanoparticle solution (11.0 mg/mL) was determined by lyophilizing a part of the stock solution.

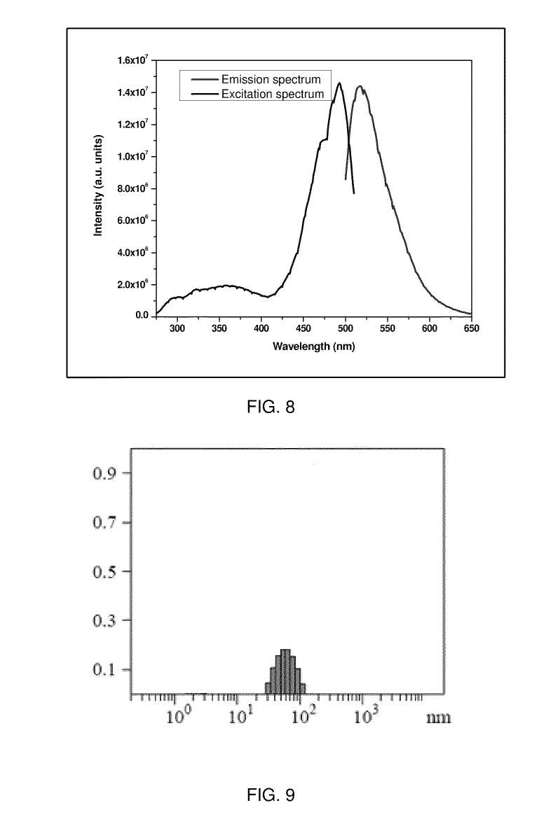

Fluorescence excitation and emission spectra of the FITC labeled BCNPs were recorded on a SPEX Nanolog (HORIBA Jobin Yvon) spectrofluorometer. FIG. 8 shows the fluorescence excitation (recorded at 519 nm emission) and emission spectra (recorded at 490 nm excitation) of the FCNPs recorded in DI water showed characteristic peaks of FITC.

A JEOL JEM 1011 100 kV transmission electron microscope (TEM) was used to characterize particle size. TEM sample was prepared by placing a drop of the BCNPs on a carbon coated copper grid (400 mesh size) followed by air drying. The particle size distribution (histogram) presented in the TEM image is from the measured particle sizes of about 75 randomly selected particles from the TEM image. BCNPs size and zeta potential in DI water were measured using the Malvern Zeta Sizer (model: NanoZS) Dynamic Light Scattering (DLS) instrument. FIG. 9 shows the particle size distribution in the ranges of 60 nm.

All MRI measurements were performed using a 4.7T Bruker Avance MR scanner. Phantom MR relaxivities were performed with serial dilutions of 10 mg/ml stock solution of BCNPs with NanoPure ddH₂O. The varying nanoparticle concentrations of 10, 5, 2.5, 1.25 and 0.625 mg/ml were loaded into Kwik-Fil™ TW150-4 capillary tubes (World Precision Instruments, Inc, Sarasota, Fla.). Prior to imaging, all samples were placed together inside a water-filled FACS tube (BD Falcon, Flanklin Lakes, N.J.) to minimize susceptibility effects from the surrounding air.

Mouse monocytes/macrophage J774 cells were suspended in DMEM complete medium (Dulbecco's modified Eagle's medium (DMEM) (GIBCO, Grand Island, N.Y.) supplemented with 10% fetal bovine serum (Summit Biotechnology, Ft. Collins, Colo.), 1% glutamax (GIBCO), 1% penicillin/streptomycin (GIBCO)), incubated at a density of 5×10⁵ cells/ml in 10 cm culture dishes at 37° C. and 5% CO₂. Culture media was replaced 24 h after plating, and the cells were allowed to attach and grow to confluency. Cells were then washed with fresh media, counted and replated at a density of 1×10⁶ cells/ml in DMEM complete medium in a 6-well culture dish. Cells were allowed to attach to the wells (2-3 h) before 100 μg/ml BCNPs were added to the wells and incubated overnight. The next day, label-containing media was aspirated off and the attached cells were washed with fresh media before being scraped up. Next, the cells were counted and re-suspended in fresh media at a density of 2×10⁸ cells/ml. Finally 20 μl, containing 4×10⁶ cells, were seeded into custom pre-made 1% Agarose well phantoms (Ultra-Pure agarose, Invitrogen, Carsbad, Calif.). The cell phantoms were kept on ice until the time of imaging.

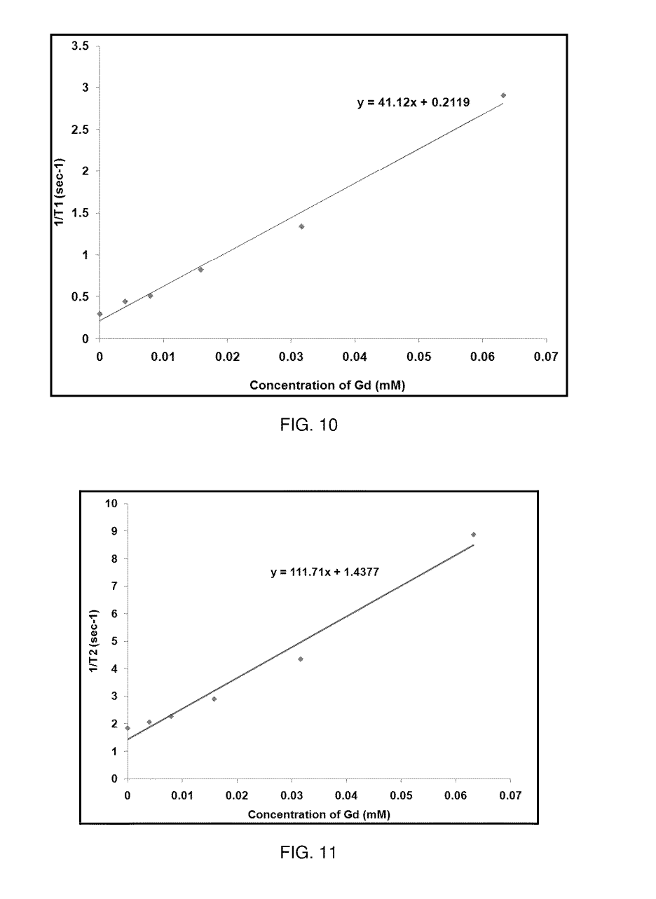

All MR relaxivity data was acquired and analyzed using Paravision software (PV3.02 Bruker Medical). For measuring T₁ relaxation times, axial spin-echo (SE) scan sequences were recorded with TE=7.094 ms, matrix size=128×64, FOV=2.5×1.25 cm², Spectral width=50 kHz, one signal average, 1 mm slice thickness and varying TR values of 11, 6, 3, 1.5, 0.75, 0.5, 0.25, 0.125, 0.075 and 0.05 s. For T₂ relaxation measurements, axial T₂-weighted single-slice multi-echo images were obtained with TR=11 s, TE=7.12 ms ΔTE=7.12 ms (60 echoes), matrix size=128×64, FOV=2.5×1.25 cm², spectral width=50 kHz, one signal average and a 1 mm slice thickness. T₁ and T₂ maps were generated assuming a monoexponential signal decay and by using a non-linear function, least-squares curve fitting on the relationship between changes in mean signal intensity within a region of interest (ROI) to TR and TE. T₁ and T₂ relaxation times (s) for the BCNPs were then derived by ROI measurements of the test samples converted into R₁ and R₂ relaxation rates (1/T₁,2 (s⁻¹)). Finally, R_{1,2} values were plotted against the concentration of Gd on the nanoparticle and r₁ and r₂ (mM⁻¹ s⁻¹) relaxivities were obtained as the slope of the resulting linear plot in FIGS. 10-11.

Briefly, J774 cells were harvested and seeded on a glass bottom culture dishes (No. 1.5) coated with poly-D-lysine (MatTek Corporation, MA) at a cell density of 1 million cells in 2 mL of cell culture medium. The next step was labeling the cells with the chitosan nanoparticles following the same procedure indicated in the materials and methods section. 24 h later, the cells were washed 2 times with PBS to remove the unbound nanoparticles. Afterwards, the same cells were labeled with a carbocyanine membrane dye (1,1-dioctadecyl-3,3,3′,3′-tetramethylindocarbocyanine perchlorate, Dil) at a concentration of 8 μm in PBS for 15 min into the cell incubator. Finally, the cells were washed 2 times with PBS to remove the excess of dye before they were maintained in 2 mL of PBS with 10% FBS.

Once the cells were labeled, confocal images were taken with a Leica TCS SP2 AOBS confocal laser mounted on a Leica DM IRE2 inverted microscope. This microscope is supplied with four lasers. Specifically, the argon laser line at 488 nm was used for excitation of BCNPs, and the emission wavelength for collection of data was 500-535 nm. For imaging of Oil, the He—Ne laser line at 543 nm was used for excitation and the data were collected over an emission range of 555-600 nm.

While various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims.

The invention claimed is:

1. A method for synthesizing water-dispersible chitosan-based nanoparticles comprising:
obtaining a first water-in-oil (MO) microemulsion comprising an oil, a surfactant, and an aqueous phase comprising a chitosan polymer;
obtaining a second microemulsion comprising an oil, a surfactant, and an aqueous phase comprising a carboxyl group-containing compound;
reacting components of the first and second microemulsions for a time sufficient to form the water-dispersible chitosan-based nanoparticles; and
recovering the water-dispersible chitosan-based nanoparticles from the reacted first and second microemulsion components, the water-dispersible chitosan based nanoparticles having an average particle size of about 100 nm or less.

2. The method of claim 1, wherein at least one of the first microemulsion or the second microemulsion further comprises a co-surfactant, and wherein the co-surfactant comprises n-hexanol.

3. The method of claim 1, wherein the carboxyl group-containing compound comprises a dicarboxylic acid, a polycarboxylic acid, a carboxyl group-containing polymer, a dicarboxylic compound, a polycarboxylic compound, or combinations thereof.

4. The method of claim 1, wherein the carboxyl group-containing compound comprises activated tartaric acid, and wherein the activated tartaric acid is prepared by reacting tartaric acid with N-hydroxysuccinimide (NHS) and a 1-ethyl-3-(3-dimethylaminopropyl carbodiimide hydrocholoride) (EDC) coupling agent.

5. The method of claim 1, further comprising bonding an imaging agent to the chitosan polymer within the aqueous phase of the first microemulsion.

6. The method of claim 5, wherein the imaging agent comprises a fluorophore, and wherein the fluorophore comprises at least one of fluorescent dye, a quantum dot, a bioluminescence agent, or combinations thereof.

7. The method of claim 1, further comprising bonding a target-specific ligand to the chitosan polymer, the ligand having an affinity for a predetermined molecular target.

8. The method of claim 1, wherein the oil comprises cyclohexane, and wherein the surfactant is a non-ionic surfactant.

9. The method of claim 1, wherein the recovering is done by adding ethanol to the reacted first and second microemulsions, and wherein the method further comprises suspending recovered nanoparticles in a fluid carrier and separating aggregated nanoparticles from monodispersed nanoparticles after the suspending.

10. The method of claim 1, wherein the chitosan polymer comprises a first chitosan polymer and a second chitosan polymer, and further comprising bonding a fluorophore to the first chitosan polymer and bonding a paramagnetic chelate having a paramagnetic ion bound therein to the second chitosan polymer such that the recovered nanoparticles are effective as a bimodal agent that is fluorescent as well as paramagnetic.

11. A water-dispersible chitosan-based nanoparticle comprising a cross-linked chitosan polymer having an imaging agent bonded thereto, wherein the chitosan nanoparticle has a particle size of about 100 nm or less.

12. The chitosan-based nanoparticle of claim 11, wherein the chitosan-based nanoparticle has a zeta potential of at least about +28 mV.

13. The chitosan-based nanoparticle of claim 11, wherein the chitosan-based nanoparticle has a particle size of about 60 nm or less.

14. The chitosan-based nanoparticle of claim 13, wherein the chitosan-based nanoparticle has a particle size of about 15 nm to about 35 nm.

15. The chitosan-based nanoparticle of claim 11, wherein the chitosan polymer is cross-linked with tartaric acid.

16. The chitosan-based nanoparticle of claim 11, further comprising a target-specific ligand bonded to the nanoparticle, wherein the ligand has a binding affinity for a predetermined molecular target.

17. The chitosan-based nanoparticle of claim 16, wherein the ligand is selected from one of an aptamer, a peptide, an oligonucleotide, folic acid, an antigen, an antibody, and combinations thereof.

18. The chitosan-based nanoparticle of claim 16, wherein the predetermined molecular target is associated with a cancer cell, a leukemia cell, an acute lymphoblastic leukemia T-cell, or combinations thereof.

19. The chitosan-based nanoparticle of claim 11, wherein the imaging agent comprises a fluorophore.

20. The chitosan-based nanoparticle of claim 11, wherein the imaging agent comprises a paramagnetic chelate having a paramagnetic ion bound therein such that the chitosan nanoparticle is effective as an MRI contrast medium.

21. The chitosan-based nanoparticle of claim 20, wherein the paramagnetic ion is selected from at least one of gadolinium, dysprosium, europium, or combinations thereof.

22. The chitosan-based nanoparticle of claim 11, wherein the imaging agent comprises a fluorophore and a paramagnetic chelate having a paramagnetic on bound therein such that the nanoparticle is effective as a bimodal agent that is fluorescent as well as paramagnetic.

23. An in vivo imaging method comprising:
administering to a subject a plurality of chitosan-based nanoparticles, wherein at least some of the chitosan-based nanoparticles comprise a chitosan polymer having an imaging agent bonded thereto, and wherein the chitosan-based nanoparticles have an average particle size of about 100 nm or less; and
detecting a presence of the chitosan nanoparticles.

24. The method of claim 23, wherein the imaging agent comprises at least one of a fluorophore or a paramagnetic chelate having a paramagnetic ion bound therein.

25. The method of claim 23, wherein the chitosan polymer comprises a mixture of fluorescent-labeled chitosan and chitosan linked with a paramagnetic chelate having a paramagnetic ion bound therein so that the nanoparticles are effective as a bimodal agent which is fluorescent as well as paramagnetic.

26. The method of claim 23, wherein the chitosan-based nanoparticles further comprise a target-specific ligand to the chitosan polymer, wherein the ligand is specific for a predetermined molecular target.

27. The method of claim 23, wherein the imaging agent comprises at least one of a bioluminescence agent or a radioisotope.
